(19) United States
(12) Patent Application Publication
(10) Pub. No.: US 2024/0382448 A1
(43) Pub. Date: Nov. 21, 2024

(54) METHODS FOR TREATING CORONAVIRUS INFECTION

(71) Applicant: Michael Rabinoff, Wesley Chapel, FL (US)

(72) Inventor: Michael Rabinoff, Wesley Chapel, FL (US)

(21) Appl. No.: 18/578,549

(22) PCT Filed: Jul. 6, 2022
(86) PCT No.: PCT/US2022/036227

§ 371 Date: Jan. 11, 2024

Related U.S. Application Data
(60) Provisional application No. 63/221,411, filed on Jul. 13, 2021.

(51) Int. Cl. A61K 31/357; A61K 31/352; A61K 38/18; A61K 45/06; A61P 31/14

(52) U.S. Cl. CPC A61K 31/357; A61K 31/352; A61K 38/1841; A61K 45/06; A61P 31/14

(57) ABSTRACT

Methods for treating a coronavirus infection in a subject, which include administering to the subject a therapeutically effective amount of an agent selected from one or more of artesunate, dihydroartemisinin, isorhamnetin, lycorine, (+)-taxifolin, baicalin, tashinone I, and desethylamodiaquine. Pharmaceutical compositions including in a pharmaceutically acceptable carrier, two or more agents selected from the group consisting of artesunate, dihydroartemisinin, isorhamnetin, lycorine, (+)-taxifolin, baicalin, tashinone I, desethylamodiaquine, artemisinin, nirmatrelvir and ritonavir, nirmatrelvir, ritonavir, baicalein, rhamnetin, (−)-taxifolin, and cryptotanshinone.

### CROSS-REFERENCE TO RELATED APPLICATIONS

This application claims benefit of priority to U.S. patent application No. 63/221,411, filed Jul. 13, 2021; the entire content of which is herein incorporated by reference in its entirety.

### TECHNICAL FIELD

The invention relates generally to the field of virology, infectious disease, and medicine. More specifically, the invention relates to methods for treating coronavirus infections in humans.

### BACKGROUND OF THE INVENTION

In humans, coronavirus causes respiratory infections, which are typically mild but can be lethal in rare forms such as SARS (severe acute respiratory syndrome)-CoV, MERS (Middle East Respiratory Syndrome)-CoV, and SARS-CoV-2 (COVID-19). In fact, there have been over 500 million documented covid-19 coronavirus cases, which have resulted in more than 6 million deaths. Coronaviruses are transmitted by aerosols of respiratory secretions, by fecal-oral route, and by mechanical transmission.

Structurally, coronavirus has a nucleocapsid of helical symmetry. The envelope of coronavirus carries three glycoproteins, namely, a spike protein which is for receptor binding and cell fusion, an envelope protein, and a membrane protein. Entry of coronavirus into a human cell occurs via two pathways, an endocytic pathway and a TMPRSS2-mediated surface pathway. Virus replication then occurs in the cell's cytoplasm. Most viral growth occurs in epithelial cells and seems to be primarily localized in the epithelium of the upper respiratory tract.

Current treatments for covid-19 include oral antiviral therapeutics, such as nirmatrelvir with ritonavir (PAXLOVID) and molnupiravir (LAGEVRIO), the intravenous infused antiviral therapeutic remdesivir, and the intravenously injected monoclonal antibody bebtelovimab. However, there remains the need for additional therapies for the treatment of covid-19 and other coronavirus diseases and infections.

### BRIEF SUMMARY OF THE INVENTION

The invention is directed to methods for treating coronavirus infection in a subject, which includes administering to the subject a therapeutically effective amount of an agent selected from one or more of artesunate, dihydroartemisinin, isorhamnetin, lycorine, (+)-taxifolin, baicalin, tashinone I, and desethylamodiaquine. In some embodiments, the subject is suffering from a coronavirus disease selected from the group consisting of severe acute respiratory syndrome (SARS), Middle East Respiratory Syndrome (MERS), and coronavirus disease 2019 (COVID-19).

In some embodiments, only one therapeutic agent is administered; however, in other embodiments at least two of the agents are administered.

In some embodiments, a therapeutically effective amount of one or more additional agents is also administered. In some embodiments, the additional agent down regulates cytokine production, optionally TGF-beta. In some embodiments, the additional agent reduces or inhibits inflammation or pulmonary fibrosis in response to viral infection. In some embodiments, the additional agent is selected from artemisinin, artesunate, and dihyroartemisinin. In some embodiments, the additional agent is selected from camostat, remdesivir, molnupiravir, AT-527, artemisinin, nirmatrelvir and ritonavir (PAXLOVID), nirmatrelvir, ritonavir, baicalein, rhamnetin, (−)-taxifolin, and cryptotanshinone

In a related aspect of the invention, use of an agent for the treatment of coronavirus infection in a subject is provided, characterized in that the agent is selected from one or more of artesunate, dihydroartemisinin, isorhamnetin, lycorine, (+)-taxifolin, baicalin, tashinone I, desethylamodiaquine

In another related aspect, a pharmaceutical composition is provided, which includes in a pharmaceutically acceptable carrier, two or more agents selected from the group consisting of artesunate, dihydroartemisinin, isorhamnetin, lycorine, (+)-taxifolin, baicalin, tashinone I, and desethylamodiaquine.

In still another related aspect of the invention, a pharmaceutical composition is provided, which includes, in a pharmaceutically acceptable carrier, a first agent selected from the group consisting of artesunate, dihydroartemisinin, isorhamnetin, lycorine, (+)-taxifolin, baicalin, tashinone I, and desethylamodiaquine; and a second agent that is different from the first agent and is selected from the group consisting of artesunate, dihydroartemisinin, isorhamnetin, lycorine, (+)-taxifolin, baicalin, tashinone I, desethylamodiaquine, camostat, remdesivir, molnupiravir, AT-527, artemisinin, nirmatrelvir and ritonavir (PAXLOVID), nirmatrelvir, ritonavir, baicalein, rhamnetin, (−)-taxifolin, and cryptotanshinone.

### BRIEF DESCRIPTION OF THE DRAWINGS

FIGS. 1A-1C depict TABLE 1, which discloses agents for use in the treatment of coronavirus in a subject; the listed agents are provided with suggested activity together with proposed mechanism of action.

FIG. 2 is a chart summarizing exemplary results from cytopathic effect (CPE) assays of antiviral agents against coronavirus infection in Vero 76, Caco-2, and Calu-3 cells.

FIGS. 3A-3D are charts summarizing results from virus yield reduction (VYR) assays against coronavirus in Vero 76 and Caco-2 cells. Data collected using visible spectrum is designated VIS and data collected using near infrared is designated NR.

FIG. 4 is a chart and graph comparing inhibition of coronavirus infection and toxicity between cells treated with different concentrations of artesunate and cells treated with different concentrations of remdesivir.

FIG. 5 is a chart and graph comparing inhibition of coronavirus infection and toxicity between cells treated with different concentrations of dihydroartemisinin and cells treated with different concentrations of remdesivir.

FIG. 6 is a chart and graph comparing inhibition of coronavirus infection and toxicity between cells treated with different concentrations of isorhamnetin and cells treated with different concentrations of remdesivir.

### DETAILED DESCRIPTION

The invention provides methods for treating coronavirus infection in a subject and pharmaceutical compositions for the treatment of coronavirus infection. As used herein the term “coronavirus” refers to the family of viruses designated as coronavirus by the U.S. National Institute of Health (NIH) and includes but is not limited to SARS-CoV, which causes severe acute respiratory syndrome (SARS); MERS-CoV, which causes Middle East respiratory syndrome (MERS); and SARS-CoV-2, which causes coronavirus disease 2019 (COVID-19). By “treating coronavirus infection” it is meant that the agent is administered prior to viral infection or after viral infection has occurred.

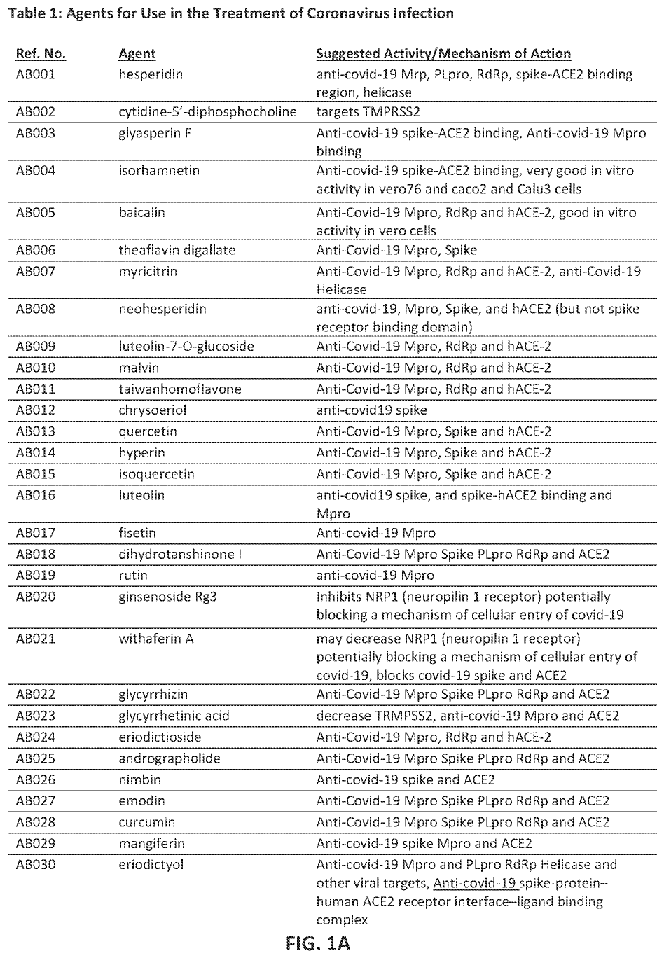

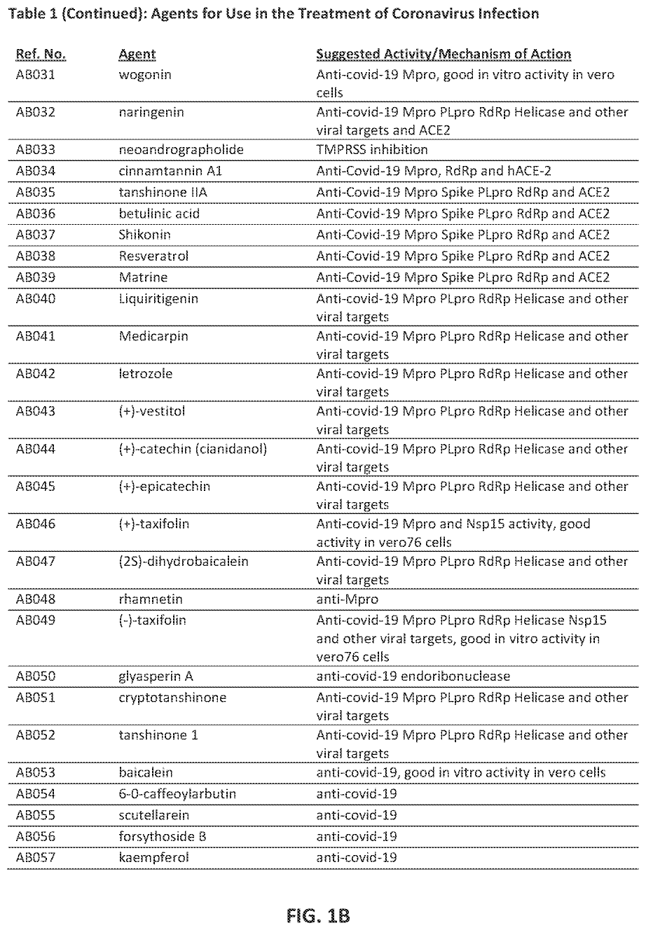

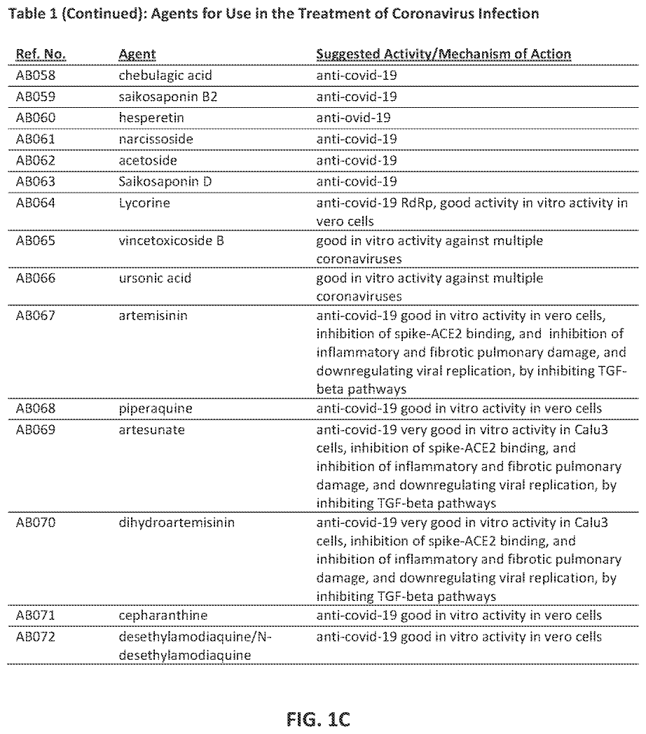

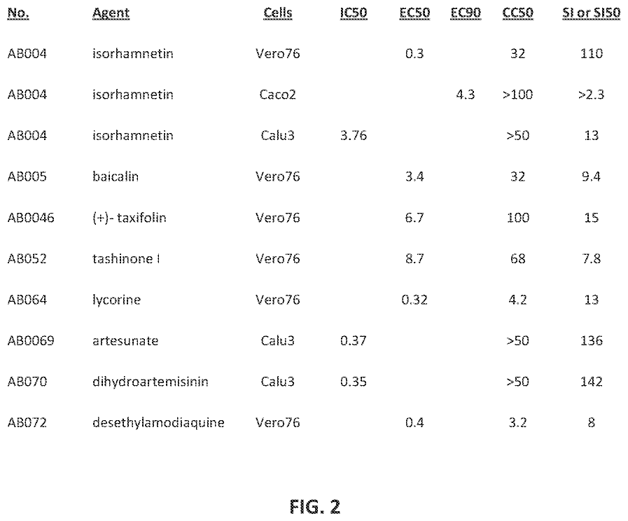

Testing described herein has utilized the non-clinical and pre-clinical services program offered by the National Institute of Allergy and Infectious Diseases. Antiviral agents described herein were identified by analyzing protein structures and functional elements of covid-19 and relevant structural and functional elements in humans. Secondary structures were modeled and screened against a library of potential antiviral compounds to assess potential interactions via computational molecular docking analysis in addition to virtual ADME studies and virtual toxicology analysis. Various forms of artificial intelligence and machine learning were also used, including generative adversarial network analysis. FIGS. 1A-1C provide table of agents that showed positive results together with their suggested activity or mechanism of action. A subset of hits was subsequently tested in cellular models. From these cellular models, FIG. 2 depicts a representative summary of results showing agents with an SI or SI50 (calculated by dividing CC50 by EC50 or by dividing CC50 by EC90) of about 8 or greater, which cellular testing shows to be most effective in the protection of cells exposed to coronavirus. The agents from FIG. 2 include artesunate, dihydroartemisinin, isorhamnetin, lycorine, (+)-taxifolin, baicalin, tashinone I, and desethylamodiaquine.

As will be described in more detail in the paragraphs that follow, a preferred technical approach is to administer one or more agents that alone or in combination target at least two different stages of the coronavirus life cycle. In some embodiments, the one or more agents target two or more of viral attachment, penetration, uncoating, biosynthesis, maturation and release. In other embodiments, the one or more agents target one or more stages of the coronavirus life cycle and act to improve host defense or host response against viral infection. In some embodiments pulmonary fibrosis or inflammation is reduced, treated, or prevented after administration of the one or more agents.

Most preferably, the one or more agents have multiple targets selected from functional and structural elements of the coronavirus and coronavirus life cycle and those that improve host defense or host response to viral infection. Without being bound by theory, administering one or more agents that target multiple stages of the viral life cycle will be effective at blocking drug combination resistance that can occur by viral mutation. Over 12,700 known mutations have been identified across millions of coronavirus infections, some of which might further mutate to adapt to single agent compositions. Moreover, by combining agents having multiple targets, it is believed toxicity can be lowered and effectiveness of the pharmaceutical composition increased.

When including an additional agent that improves host defense, one approach is to downregulate the production of cytokines, such as by way of transforming growth factor beta (TGF-beta) pathways. TGF-beta signaling can lead to several complications including inflammation and pulmonary fibrosis. As such, by providing an additional agent that inhibits or down regulates cytokines, a cytokine storm resulting from viral infection can be reduced, thereby improving host defense while the primary antiviral agent(s) targets the coronavirus itself.

Turning back to the technical approach of targeting the coronavirus life cycle, in some embodiments at least one administered agent affects the coronavirus spike protein. There are two entry pathways for covid-19, an endocytic pathway, and a TMPRSS2-mediated surface pathway. It is now known that the SARS-CoV2 spike protein can interact with the angiotensin-converting enzyme 2 (ACE2) cellular receptor, which can lead to rapid endocytosis. As such, in some embodiments, at least one administered agent interferes with coronavirus spike protein binding to ACE2. It is believed that targeting the spike protein or the ACE2 receptor reduces endocytosis. Thus, in some embodiments at least one administered agent targets the interaction between the coronavirus spike protein and ACE2. In some embodiments at least one administered agent interferes with (e.g. inhibits or reduces) spike protein binding at the ACE2 binding site or allosterically.

Agents that may be used to target ACE2-spike binding include hesperidin, glyasperin F, isorhamnetin, chyrsoeriol, quercetin, hyperin, isoquercetin, luteolin, dihydrotanshinone I, tanshinone IIA, betulinic acid, shikonin, resveratrol, matrine, artenusate, and dihydroartemisinin. Others can include compounds from Lonicerae flos and mori folium, Saikosaponons U and V and others, bupleurum compounds, heteromorpha, cfophunart, phylloambllein 7, swerta xanthons, neohesperdin, hersperidin, 18beta-glycyyhetinic acid, stigmasterol, indigo, beta-sitosterol, luteolin, quercetin, naringenin, dihydrotanshinone I (honeysuckle Mulberry).

Since ACE2 and TMPRSS2 are individually expressed in some human cell types and co-expressed in other cell types, the approach of simultaneous inhibition of virus entry through the endocytic pathway and the surface fusion pathway mediated by TMPRSS2 may have better antiviral effect. To this end, it is believed that inhibiting binding to TMPRSS2 or downregulating TMPRSS2, such as by using TMPRSS2 inhibitors, will be a useful in the treatment against coronavirus infection. Accordingly, in some embodiments at least one administered agent downregulates TMPRSS2. In some embodiments at least one administered agent interferes with TMPRSS2 binding, either by interaction at the binding site or allosterically. Agents that may be used to target TMPRSS2 include camostat, cytidine-5′diphosphocholine, glycyrrhetinic acid, and neoandrographolide. Other agents include phyllaembicine E, neoandrographolide, kouitchside I and D, licoflavanol, cosmosiinfrom, neohesperidin, mangostinfrom, excoelarsat, picetraminol, phyllaembilic G7, geniposide (poor gi absorption), schiphenin A, dictyospaeric acid A, cytidine-5′-diphosphocoline, 5′-methoxyhydnocarpin D, curtisian L, microcarpin, (−)-epicatechin e-O-(3′-O-methyl)gallate, isogemichalone B, fuscaxanthone A, orthosiphonone D, 7-hydroxy-14-deoxywithanolide, 6S,9R-roseoside, and camostat.

In some embodiments, the method of treating coronavirus infection includes administering to the subject a therapeutically effective amount of a first agent that affects binding between the spike protein and ACE2, and a second agent that downregulates TMPRSS2 or inhibits binding to TMPRSS2.

In some embodiments at least one administered agent targets or affects Mpro (main protease). In some embodiments this agent is an Mpro inhibitor. Mpro is a viral encoded cysteine protease that has as a preference for a glutamine residue at the P1 site. Thus, most Mpro inhibitors contain either 2 pyrrolidone or 2-piperidinone at the P1 site as a memetic of the glutamine residue. Non-limiting examples of agents that may have an effect on Mpro and which may be used in the pharmaceutical composition include glyasperin F, baicalin, theaflavin digallate, myricitrin, neohesperidin, luteolin-7-O-glucoside, malvin, taiwanhomoflavone, quercetin, hyperin, isoquercetin, luteolin, fisetin, dihydrotanshinone I, rutin, glycyrrhizin, glycyrrhetinic acid, eriodictioside, andrographolide, nimbin, emodin, curcumin, mangiferin, eriodictyol, wogonin, naringenin, cinnamtannin A1, tanshinone IIA, betulinic acid, Shikonin, Resveratrol, Matrine, Liquiritigenin, Medicarpin letrozole, vestitol, catechin (cianidanol), epicatechin, taxifolin, dihydrobaicalein, rhamnetin, glyasperin A, cryptotanshinone, tanshinone 1. Others can include acetoside, crocin, digitoxigen, beta-eudesmol, luteolin, licoisoflavone B, quercetin, EGCG, fisetin, isolicoflavanol, semilicoisoflavanone-B, quercetin-7-rhamnoside, pentapeptide A, ligustrazine, salvianolic acid B, coumaroyltyramine, desmethoxyreserpine, dihomo-c-linolenic acid, lignan, n-cis-feruloyltyramine, sugiol, celestrol, pristimern, tingenone, iguesteru, dihydrocelestrol, andrographide derivatives, betulonal, isodecortinol, cerevisterol, icohesperidil, daidzein, aloe-emodin, artemisinin, 10-Hydroxyusambarensine, Cryptoquindoline, 6-Oxoisoiguesterin, 22-Hydroxyhopan-3-one, Cryptospirolepine, Isoiguesterin and 10-Hydroxyusambarensine, cryptoquindoline, the terpenoids 6-Oxoisoiguesterin and 22-Hydroxyhopan-3-one, hypericin, cyanidin-3-glycoside, glabridin, alpha ketoamine 11r, pseudohypericin, licorice glycoside E, naringenin, robinin, kaempferol, isorhamnetin, irisolidone, 2E-7-hydroxy-2-(4-hydroxyphenyl) chroman-4-ketone B, aster pentapeptide A, ligustrazine, salvianolic acid B.

In some embodiments at least one administered agent targets or affects the RNA-dependent RNA polymerase (RdRp). RdRp is a viral enzyme for viral RNA replication in host cells. Since RdRp is a viral enzyme with no host cell homologs, selectively targeting RdRp with inhibitor is believed to have few if any off-target effects when used to treat human subjects or mammalian cells. In some embodiments, the agent is an RdRp inhibitor. Exemplary RdRp inhibitors that may be included in the pharmaceutical composition include favipiravir, ribavirin, galidesivir, and theaflavin. Other exemplary agents include gnidicin, gniditrin, betulonal, Andrographis, viola, and swerta compounds, theaflavin, 3,3′ dio-gallate, and andrographolide derivatives. Still others include luteolin-7-O-glucoside, malvin, taiwanhomoflavone, dihydrotanshinone I, glycyrrhizin, eriodictioside, andrographolide, emodin, curcumin, naringenin, cinnamtannin A1, tanshinone IIA, betulinic acid, Shikonin, resveratrol, Matrine, Liquiritigenin, Medicarpin, letrozole, vestitol, catechin (cianidanol), epicatechin, dihydrobaicalein, taxifolin, cryptotanshinone, tanshinone 1, and Lycorine. Remdesivir and molnupiravir are also believed to target RdRp. Other agents affecting replication can also be included. Amont those include AT-527, which inhibits viral replication by interfering with RNA polymerase.

In some embodiments, at least one administered agent includes or also includes one or more agents that inhibit Mpro, RdRp and ACE2 (other than the ACE2 binding region), including Chrysanthemin, myritilin, myricitrin, malvin, asperlicin C, casameridin, epicatechin derivatives, gambinin B2, 6-hydroxybenzoylhyperin, gambinin A3, cyanidin, lactucopiricin, trictinin, chrysin-7-0-derivatives, baicalin, ute-olin-7-glucoside, hyperin, isoquercetin, taiwanhomoflavone A, lactucopicrin-15-oxylate (and biorobin, pedunculagin, and afzelin with these 3 having less anti-ACE2 activity). Others include thymol, limonene, p-cymene, gamma terpinene, allyl disulfide, allyl trisulfite, allyl methyl trisulfide, diallyl tetrasulfide, trisulfide 2-propenyl, kaempherol 3-o-rutinoside, andrographolide and 1-derivatives, neoadrographolide, and xanthones from swerta.

In some embodiments, at least one administered agent targets or affects the papin-like protease (PLpro). PLpro is an essential coronavirus enzyme that is required for processing viral polyproteins to generate a functional replicase complex and enable viral spread. PLpro is also implicated in post-translational modification of host proteins as an evasion mechanism against host antiviral immune responses. As such, inhibition of PLpro is predicted to block coronavirus replication. Exemplary agents that can be included in the pharmaceutical composition include those targeting PLpro include ginger ketophenol, ginkgol alcohol, ferulic 7 acid, coumaroyltyramine, cryptotanshinone, moupinamide, n-cis-feruloyltyramine, quercetin. Platycodon D, baicalin, surgetriol-3,9-diacetate, phaitantrin D, 2,2-Nn(3, n-dolyl) 3 indole, catechin, EGCG, gingerketophenol, ginkgol alcohol, ferulic acid. Other agents that may target PLpro include hesperidin, dihydrotanshinone I, glycyrrhizin, andrographolide, emodin, curcumin, naringenin, tanshinone IIA, betulinic acid, Shikonin, Resveratrol, Matrine, Liquiritigenin, Medicarpin, letrozole, vestitol, catechin (cianidanol), epicatechin, dihydrobaicalein, taxifolin, cryptotanshinone, and tanshinone 1.

In some embodiments, at least one administered agent targets or affects non-structural protein 1 (Nsp1). Nsp1, also referred to as the host shutoff factor, suppresses host innate immune functions. As such, inhibiting interaction with Nsp1 would prevent or reduce this host shutoff. Nonlimiting examples of agents that target or affect Nsp1 include fumarate.

In some embodiments, at least one administered agent targets or affects the Nsp13 Helicase. Nsp13 is critical for viral replication and is the most conserved nonstructural protein within the coronavirus family. Accordingly, Nsp13 is a promising target. Exemplary agents targeting helicase include alphaglucosyl hesperidin, hesperidin, rutin, quercetaguin, glycopranosyl and xanthones, homovitexin, kouitchenside D, swerta compounds, triptexanthoside D, and phyllamblinol. Further, agents with effect against SARS-CoV but not yet tested against SARS-CoV2, include for anti-helicase, scutellarein, silvestrol, saikosponin B2, caffeic acid, and against SARS-CoV PLpro, psoralidin, isobavachalcone, neobavachalcone, griffithsin and other lectins.

Gancao compounds and derivatives that may be used to fight coronavirus infection include quercetin, 5,7,2′,6′ tetrahydroxyflavine, I4 kaempferol, 4′-hydroxywogonin, ganhuangenin, I5 baicalin, gancolin, norwogonin.

In some embodiments, at least one administered agent targets or affects Heat Shock Protein A5 (HSPA5), also termed GRP78 or BiP. HSPA5 substrate-binding domain β (SBDβ) is a recognition site for the SARS-CoV-2 spike. These can be targeted using phytoestrogens, such as Diadiazin, Genistein, Formontein, and Biochanin A, chlorogenic acid, linolenic acid, palmitic acid, caffeic acid, caffeic acid phenethyl ester, hydroxytyrosol, cis-p-Coumaric acid, cinnamaldehyde, thymoquinone, and estrogens, progesterone, testosterone, and cholesterol.

The methods can also include administration of one or more agents that target or affect other structural targets of coronavirus, such as natural flavonols, licoflavanol and glycyrriza compounds, cosmoii neohesperidin, mangostinfrom, kutchenside D, excoecariatoxin, phyllaemlingy, and piceatannol.

In some embodiments, at least one administered agent is a virulence targeting enhancing agent, such as vitex neguno compounds, andrographolide derivatives and xanthones from swerta.

As already introduced, the preferred approach for treating coronavirus infection in a subject uses a multi-target approach. A multi-target approach can be accomplished using a pharmaceutical composition containing a single agent targeting different stages of viral infection and replication or two or more agents in combination. For example, hesperidin targets Mpro, RdRp and the ACE2-spike binding region, and thus inhibits all three (3) viral functions and thus the pharmaceutical composition may contain hesperidin as an sole agent or one, two or more agents in a multitarget approach.

As another example, dihydroartemisinin may be administered for inhibition of spike binding, inhibition of inflammatory and fibrotic pulmonary damage and down regulation of viral replication by inhibiting TGF-beta pathways. However, treatment may be improved by adding additional agents to dihydroartemisinin. For example, the addition of one or more agents selected from isorhamnetin (spike-ACE2 binding), lycorine (RdRp, N protein), taxifolin (as racemic taxifolin or (+)-taxifolin or (−)-taxifolin: Mpro, Nsp15), camostat (TMPRSS2), remdesivir (RdRp), molnupiravir (RNA mutagenesis by RdRP), AT-527 (inhibit viral replication), baicalin (Mpro, RdRp and ACE-2), baicalein (anti-covid-19), desethylamodiaquine (anti-covid-19), and tanshinone 1 (Mpro PLpro RdRp Helicase and other viral targets) to dihdyroartemisinin may be more effective.

As still another example, artesunate may be administered for inhibition of spike-ACE2 binding, inhibition of inflammatory and fibrotic pulmonary damage, and downregulating viral replication by inhibiting TGF pathways; however, the treatment may be improved by providing in addition to artesunate, one or more agents selected from isorhamnetin (spike-ACE2 binding), lycorine (RdRp, N protein), taxifolin (as racemic taxifolin or (+)-taxifolin or (−)-taxifolin: Mpro, Nsp15), camostat (TMPSSR2), remdesivir (RdRp), molnupiravir (RNA mutagenesis by RdRP), AT-527 (inhibit viral replication), baicalin (Mpro, RdRp and ACE-2), baicalein (anti-covid-19), desethylamodiaquine (anti-covid-19), and tanshinone 1 (Mpro PLpro RdRp Helicase and other viral targets).

As still another example isorhamnetin is administered to target spike-ACE2 binding; however, the treatment may be improved by adding one or more agent selected from lycorine (RdRp, N protein), taxifolin (as racemic taxifolin or (+)-taxifolin or (−)-taxifolin: Mpro, Nsp15), camostat (TMPSSR2), remdesivir (RdRp), molnupiravir (RNA mutagenesis by RdRP), and AT-527 (inhibit viral replication), baicalin (Mpro, RdRp and ACE-2), baicalein (anti-covid-19), desethylamodiaquine (anti-covid-19), and tanshinone 1 (Mpro PLpro RdRp Helicase and other viral targets).

As still another example, lycorine may be administered to target RdRp and N protein; however, the treatment may be improved by adding one or more agent selected from taxifolin (as racemic taxifolin or (+)-taxifolin or (−)-taxifolin: Mpro, Nsp15), camostat (TMPSSR2), remdesivir (RdRp), molnupiravir (RNA mutagenesis by RdRP), AT-527 (inhibit viral replication), baicalin (Mpro, RdRp and ACE-2), baicalein (anti-covid-19), desethylamodiaquine (anti-covid-19), and tanshinone 1 (Mpro PLpro RdRp Helicase and other viral targets).

As still another example, taxifolin (as racemic taxifolin or (+)-taxifolin or (−)-taxifolin) is administered to target Mpro and NSP15; however, the treatment may be improved by adding in addition to taxifolin, one or more agent selected from camostat (TMPSSR2), remdesivir (RdRp), molnupiravir (RNA mutagenesis by RdRP), and AT-527 (inhibit viral replication), baicalin (Mpro, RdRp and ACE-2), baicalein (anti-covid-19), desethylamodiaquine (anti-covid-19), and tanshinone 1 (Mpro PLpro RdRp Helicase and other viral targets).

In still other examples, the treatment may include administering a therapeutically effective amount of one or more of the agents listed in Table 1, which is presented in FIGS. 1A-1C, alone or in combination for the treatment of coronavirus infection in the subject. FIGS. 1A-1C list agents having positive results from testing together with proposed mechanism of action, the agents including hesperidin, cytidine-5′-diphosphocholine, glyasperin F, isorhamnetin, baicalin, theaflavin digallate, myricitrin, neohesperidin, luteolin-7-O-glucoside, malvin, taiwanhomoflavone, chrysoeriol, quercetin, hyperin, isoquercetin, luteolin, fisetin, dihydrotanshinone I, rutin, ginsenoside Rg3, withaferin A, glycyrrhizin, glycyrrhetinic acid, eriodictioside, andrographolide, nimbin, emodin, curcumin, mangiferin, eriodictyol, wogonin, naringenin, neoandrographolide, cinnamtannin A1, tanshinone IIA, betulinic acid, Shikonin, Resveratrol, Matrine, Liquiritigenin, Medicarpin, letrozole, (+)-vestitol, (+)-catechin (cianidanol), (+)-epicatechin, (+)-taxifolin, (2S)-dihydrobaicalein, rhamnetin, (−)-taxifolin, glyasperin A, cryptotanshinone, tanshinone 1, baicalein, 6-0-caffeoylarbutin, scutellarein, forsythoside B, kaempferol, chebulagic acid, saikosaponin B2, hesperetin, narcissoside, acetoside, Saikosaponin D, Lycorine, vincetoxicoside B, ursonic acid, artemisinin, piperaquine, artesunate, dihydroartemisinin, cepharanthine, and desethylamodiaquine/N-desethylamodiaquine.

### Pharmaceutical Compositions for the Treatment of Coronavirus Infection

Preferably, one or more of the above provided agents is formulated as a pharmaceutical composition for administration to a subject or mammalian cell in need thereof. Thus “pharmaceutical composition” as used throughout this document refers to an agent (e.g. a chemical or a biological compound or substance) intended for use in the medical diagnosis, cure, treatment, or prevention of disease or pathology of coronavirus infection. Within the meaning of “pharmaceutical composition” the agent is combined with a pharmaceutically acceptable carrier. The term “carrier” refers to a substance that serves as a vehicle for improving the efficiency of delivery and the effectiveness of a pharmaceutical composition. The term “pharmaceutically acceptable” when used to define the carrier, whether diluent or excipient, refers to a substance that is compatible with the other ingredients of the formulation and not substantially deleterious to the recipient thereof.

In clinical practice the pharmaceutical compositions provided herein may be administered by any conventional route, such as but not limited to orally, parenterally, or by inhalation (e.g. in the form of aerosols). In certain embodiments, the pharmaceutical compositions provided herein are administered orally and in other embodiments the pharmaceutical compositions are administered by injection or intravenous infusion.

When administered orally, the pharmaceutical composition may be formulated as a solid composition, such as tablets, pills, hard gelatin capsules, powders or granules. In these compositions, the one or more agent is mixed with one or more inert diluents or adjuvants, such as sucrose, lactose or starch. The pharmaceutical compositions can include substances other than diluents, for example a lubricant, such as magnesium stearate, or a coating intended for controlled release.

The pharmaceutical composition may be formulated as a liquid composition for oral administration, such as those including pharmaceutically acceptable suspensions, emulsions, syrups and elixirs containing inert diluents, such as water or liquid paraffin. These compositions can also include substances other than diluents, for example wetting, sweetening or flavoring products.

The pharmaceutical compositions for parenteral administration can be emulsions or sterile solutions. Pharmaceutical compositions may contain a saline carrier. Pharmaceutical compositions may be formulated with a solvent or vehicle, of propylene glycol, a polyethylene glycol, vegetable oils, in particular olive oil, or injectable organic esters, for example ethyl oleate. These compositions can also contain adjuvants, in particular wetting, isotonizing, emulsifying, dispersing and stabilizing agents. Sterilization can be carried out in several ways, for example using a bacteriological filter, by radiation or by heating. They can also be prepared in the form of sterile solid compositions which can be dissolved at the time of use in sterile water or any other injectable sterile medium.

The pharmaceutical compositions can also be aerosols. For use in the form of liquid aerosols, the pharmaceutical compositions can be stable sterile solutions or solid compositions dissolved at the time of use in sterile water, in saline or any other pharmaceutically acceptable carrier. For use in the form of dry aerosols intended to be directly inhaled, the active principle is finely divided and combined with a water-soluble solid diluent or vehicle, for example dextran, mannitol or lactose.

In furtherance of the above, typical pharmaceutical compositions and dosage forms include one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific active ingredients in the dosage form. The composition or single unit dosage form, if desired, can also contain minor amounts of wetting or emulsifying agents, or pH buffering agents.

Lactose free compositions provided herein can include excipients that are well known in the art and are listed, for example, in a variety of well-accepted pharmaceutical references. In general, lactose free compositions have an active agent, a binder/filler, and a lubricant in pharmaceutically compatible and pharmaceutically acceptable amounts. Exemplary lactose free dosage forms include an active agent, microcrystalline cellulose, pre gelatinized starch, and magnesium stearate.

Further, encompassed herein are anhydrous pharmaceutical compositions and dosage forms having one more agent, since water can facilitate the degradation of some compounds. For example, the addition of water (e.g., 5%) is widely accepted in the pharmaceutical arts as a means of simulating long term storage in order to determine characteristics such as shelf life or the stability of formulations over time. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that include lactose and at least one active agent that includes a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Further provided are pharmaceutical compositions and dosage forms that include one or more compounds that reduce the rate by which an active agent will decompose. Such compounds, which are referred to herein as “stabilizers,” include, but are not limited to, antioxidants such as ascorbic acid, pH buffers, or salt buffers.

The pharmaceutical composition's shape, and type of dosage forms provided herein will typically vary depending on their use. For example, a dosage form used in the initial treatment of viral infection may contain a larger amount of one or more of the active agents compared to a dosage form used in the maintenance treatment of the same infection. Similarly, a parenteral dosage form may contain smaller amounts of one or more of the active agents compared to an oral dosage form used to treat the same disease or disorder. These and other ways in which specific dosage forms encompassed herein will vary from one another will be readily apparent to those skilled in the art. See, e.g., Remington's Pharmaceutical Sciences, 20th ed., Mack Publishing, Easton Pa. (2000).

Generally, the ingredients of the pharmaceutical compositions are supplied either separately or mixed together in unit dosage form, for example, as a dry lyophilized powder or water free concentrate in a hermetically sealed container such as an ampoule indicating the quantity of active agent. Where the composition is to be administered by infusion, it can be dispensed with an infusion bottle containing sterile pharmaceutical grade water or saline as carrier. Where the composition is administered by injection, an ampoule of sterile water for injection or saline can be provided so that the ingredients may be mixed prior to administration.

Typical dosage of the agent provided within the pharmaceutical, may lie the range of from about 0.1 mg to about 1000 mg per day, given as a single once-a-day dose in the morning or as divided doses throughout the day taken with food. Particular dosage forms can have about 0.1, 0.2, 0.3, 0.4, 0.5, 1.0, 2.0, 2.5, 5.0, 10.0, 15.0, 20.0, 25.0, 50.0, 100, 200, 250, 500 or 1000 mg of each agent.

### Methods for Treating Coronavirus Infection

The agents and pharmaceutical compositions described throughout this document are used to treat human subjects or mammalian cells infected with or at risk of coronavirus infection. The term “treat” or “treatment” as used herein refers to ameliorating the disease or disorder that exists in a subject or cell. In another embodiment, “treat” or “treatment” includes ameliorating at least one physical parameter, which may be indiscernible by the subject. In yet another embodiment, “treat” or “treatment” includes modulating the disease or disorder, either physically (e.g., stabilization of a discernible symptom) or physiologically (e.g., stabilization of a physical parameter) or both. In yet another embodiment, “treat” or “treatment” includes delaying the onset of the disease or disorder. The term “treat” or “treatment” preferably prevents further infection of coronavirus within the subject or at least slows the continued infection of coronavirus within the subject. Non-limiting examples of the diseases that the invention is intended to treat in response to coronavirus infection include severe acute respiratory syndrome (SARS), Middle East respiratory syndrome (MERS), and covid-19.

During treatment, subjects or mammalian cells are provided with a therapeutically effective amount of the agent(s), typically in the form of a pharmaceutical composition. By “therapeutically effective amount” it is meant that the agent(s) is provided in an amount that will elicit the biological or medical response that is being sought. It will be understood by those having ordinary skill in the art to which the invention belongs that the specific dose level and frequency of dosage for any particular subject or cell may be varied and will depend upon a variety of factors including the activity of the specific active agent employed, the metabolic stability and length of action of that active agent, the age, body weight, general health, gender, diet, and the severity of the particular disease or condition being treated.

In a clinical environment, the doctor will determine which is the most appropriate dosage for the treatment and according to the age, weight, stage of the infection and other factors specific to the subject to be treated. In certain embodiments, doses are from about 1 to about 1000 mg per day for an adult, or from about 5 to about 250 mg per day or from about 10 to 50 mg per day for an adult. In certain embodiments, doses are from about 5 to about 400 mg per day or 25 to 200 mg per day per adult. In certain embodiments, dose rates of from about 50 to about 500 mg per day are also contemplated.

In certain embodiments, the dosage of the agents or pharmaceutical compositions provided herein, based on weight of the active agent, administered to prevent, treat, manage, or ameliorate a coronavirus infection, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the active agent administered to prevent, treat, manage, or ameliorate a coronavirus disorder, or one or more symptoms thereof in a subject is a unit dose of 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

Further, the amount of the active agent or composition which will be effective in the prevention or treatment of coronavirus infection or one or more symptoms thereof will vary with the nature and severity of the infection or condition, and the route by which the active ingredient is administered. The frequency and dosage will also vary according to factors specific for each subject depending on the specific therapy (e.g., therapeutic or prophylactic agents) administered, the severity of the infection, disease, or condition, the route of administration, as well as age, body, weight, response, and the past medical history of the subject. Effective doses may be extrapolated from dose-response curves derived from in vitro or animal model test systems.

Different therapeutically effective amounts may be applicable for different coronavirus infections, diseases and conditions. Similarly, amounts sufficient to prevent, manage, treat or ameliorate such infections, but insufficient to cause, or sufficient to reduce, adverse effects associated with the pharmaceutical composition provided herein are also encompassed by the dosage amounts and dose frequency schedules. Further, when a subject is administered multiple dosages of a pharmaceutical composition provided herein, not all of the dosages need be the same. For example, the dosage administered to the subject may be increased to improve the prophylactic or therapeutic effect of the pharmaceutical composition or it may be decreased to reduce one or more side effects that a particular subject is experiencing.

In certain embodiments, treatment can be initiated with one or more loading doses of an agent or pharmaceutical composition provided herein followed by one or more maintenance doses. In such embodiments, the loading dose can be, for instance, about 60 to about 400 mg per day, or about 100 to about 200 mg per day for one day to five weeks. The loading dose can be followed by one or more maintenance doses. In certain embodiments, each maintenance does is, independently, about from about 10 mg to about 200 mg per day, between about 25 mg and about 150 mg per day, or between about 25 and about 80 mg per day. Maintenance doses can be administered daily and can be administered as single doses, or as divided doses.

In certain embodiments, a dose of an agent or pharmaceutical composition provided herein can be administered to achieve a steady-state concentration of the agent in blood or serum of the subject. The steady-state concentration can be determined by measurement according to techniques available to those of skill or can be based on the physical characteristics of the subject such as height, weight and age. In certain embodiments, a sufficient amount of an agent or pharmaceutical composition provided herein is administered to achieve a steady-state concentration in blood or serum of the subject of from about 300 to about 4000 ng/ml, from about 400 to about 1600 ng/ml, or from about 600 to about 1200 ng/ml. In some embodiments, loading doses can be administered to achieve steady-state blood or serum concentrations of about 1200 to about 8000 ng/ml, or about 2000 to about 4000 ng/ml for one to five days. In certain embodiments, maintenance doses can be administered to achieve a steady-state concentration in blood or serum of the subject of from about 300 to about 4000 ng/ml, from about 400 to about 1600 ng/ml, or from about 600 to about 1200 ng/mL.

In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In certain embodiments, provided herein are unit dosages including the agent or pharmaceutical composition. Such forms are described in detail above. In certain embodiments, the unit dosage is 1 to 1000 mg, 5 to 250 mg or 10 to 50 mg active ingredient. In particular embodiments, the unit dosages includes about 1, 5, 10, 25, 50, 100, 125, 250, 500 or 1000 mg active agent. Such unit dosages can be prepared according to techniques familiar to those of skill in the art.

When using two agents as a combination therapy, dosages lower than those which have been or are currently being used to prevent or treat coronavirus infection are envisioned. In particular, combining agents can result in a synergistic effect. The recommended dosages of second agents can be obtained from the knowledge of those of skill. Thus, while much of the above has been primarily described with reference to administration of a pharmaceutical having a single active agent, the invention also includes use of two or more active agents for the treatment of a subject or cell suffering from a coronavirus infection or at risk of coronavirus infection. In furtherance of this, in some embodiments a therapeutically effective amount of at least two active agents, at least three active agents, or at least four active agents or more in a same pharmaceutical composition or in different pharmaceutical compositions is administered to a subject in need thereof. In further embodiments one, two, three, four or more active agents target one or more of coronavirus attachment, penetration, uncoating, biosynthesis, maturation or release. In some embodiments, one, two, three or four or more active agents target one or more of a spike protein, ACE2, Mpro, RdRp, PLpro, Nsp13 helicase, and TMPRSS2.

In certain embodiments, a first agent or pharmaceutical composition provided herein and a second agent are administered to a subject, for example, a mammal, such as a human, in a sequence and within a time interval such that the first agent provided herein can act together with the second agent to provide an increased benefit than each was administered otherwise. For example, the second active agent can be administered at the same time or sequentially in any order at different points in time; however, if not administered at the same time, they should be administered sufficiently close in time so as to provide the desired therapeutic or prophylactic effect. In one embodiment, the first agent or pharmaceutical composition provided herein and the second agent exert their effect at times which overlap. Each second active agent can be administered separately, in any appropriate form and by any suitable route. In other embodiments, the compound provided herein is administered before, concurrently or after administration of the second active agent.

In various embodiments, the therapies (e.g., first agent or pharmaceutical composition provided herein and the second agent) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In various embodiments, the therapies are administered no more than 24 hours apart or no more than 48 hours apart. In certain embodiments, two or more therapies are administered within the same patient visit. In other embodiments, the active agent or pharmaceutical composition provided herein and the second active agent are administered concurrently as a single composition or as two distinct compositions.

In other embodiments, the first agent or pharmaceutical composition provided herein and the second agent are administered at about 2 to 4 days apart, at about 4 to 6 days apart, at about 1 week part, at about 1 to 2 weeks apart, or more than 2 weeks apart.

In certain embodiments, administration of the same active agent may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same agent may be repeated and the administration may be separated by at least at least 1 day, 2 days, 3 days, 5 days, 10 days, 15 days, 30 days, 45 days, 2 months, 75 days, 3 months, or 6 months.

In certain embodiments, the first agent provided herein and the second agent are cyclically administered to a patient. Cycling therapy involves the administration of a first agent (e.g., a first prophylactic or therapeutic agent) for a period of time, followed by the administration of a second agent and/or third agent (e.g., a second and/or third prophylactic or therapeutic agents) for a period of time and repeating this sequential administration. Cycling therapy can reduce the development of resistance to one or more of the therapies, avoid or reduce the side effects of one of the therapies, and/or improve the efficacy of the treatment.

In certain embodiments, the first agent or pharmaceutical composition provided herein, and the second active agent are administered in a cycle of less than about 3 weeks, about once every two weeks, about once every 10 days or about once every week. One cycle can comprise the administration of a compound provided herein and the second agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In other embodiments, courses of treatment are administered concurrently to a subject, i.e., individual doses of the second agent are administered separately yet within a time interval such that the first agent provided herein can work together with the second active agent. For example, one agent can be administered once per week in combination with the other components that can be administered once every two weeks or once every three weeks. In other words, the dosing regimens are carried out concurrently even if the therapeutics are not administered simultaneously or during the same day.

The second agent can act additively or synergistically with an first agent or pharmaceutical composition provided herein. As used herein, the term “synergistic” or “synergistically” includes a combination of agents which has been or is currently being used to prevent, manage or treat a disorder, which is more effective than the additive effects of the therapies. A synergistic effect of a combination of therapies (e.g., a combination of active agents that have different mechanisms of action or act on different stages of viral infection) permits the use of lower dosages of one or more of the therapies and/or less frequent administration of the therapies to a subject in need thereof. The ability to utilize lower dosages of a therapy and/or to administer the therapy less frequently may reduce the toxicity associated with the administration of the therapy to a subject without reducing the efficacy of the therapy in the prevention or treatment of a disorder. In addition, a synergistic effect can result in improved efficacy of agents in the prevention or treatment of a disorder. Finally, a synergistic effect of a combination of therapies (e.g., a combination of active agents that target different stages of viral infection) may avoid or reduce adverse or unwanted side effects associated with the use of either therapy alone.

To this end, in some embodiments, a first agent is administered concurrently with one or more second agents in a same pharmaceutical composition. In another embodiment, an agent provided herein is administered concurrently with one or more second agents in two or more pharmaceutical compositions. In still another embodiment, a first agent provided herein is administered prior to or subsequent to administration of a second agent. Also contemplated is administration of an agent provided herein and a second agent by the same or different routes of administration, e.g., oral and parenteral. In certain embodiments, when a first agent provided herein is administered concurrently with a second agent that potentially produces adverse side effects including, but not limited to, toxicity, the second active agent can advantageously be administered at a dose that falls below the threshold that the adverse side effect is elicited.

### EXAMPLE 1

### Reduction of SARS-Cov2 Virus-Induced Cytopathic Effect (Primary CPE Assay)

Confluent or near-confluent cell culture monolayers of cells (Vero76, Caco-2 or Calu3 cells) were prepared in 96-well disposable microplates the day before testing. Cells were maintained in MEM supplemented with 5% FBS. For antiviral assays the same medium was used but with FBS reduced to 2% and supplemented with 50-μg/ml gentamicin. Compounds were dissolved in DMSO, saline or other diluent. Less soluble compounds can be vortexed, heated, and sonicated, and if they still do not go into solution, tested as colloidal suspensions. The test compounds were prepared at four serial log₁₀ concentrations, usually 0.1, 1.0, 10, and 100 μg/ml or μM. Five microwells were used per dilution: three for infected cultures and two for uninfected toxicity cultures. Controls for the experiment consisted of six microwells that were infected and not treated (virus controls) and six were untreated and uninfected (cell controls) on every plate. A known active drug was tested in parallel as a positive control drug using the same method as is applied for test compounds. The positive control was tested with every test run.

Growth media was removed from the cells and the test compound was applied in 0.1 ml volume to wells at 2× concentration. SARS-CoV2 virus (Virus ID: USA_WA1/2020), normally at ^{˜}60 CCID₅₀ (50% cell culture infectious dose) in 0.1 ml volume was added to the wells designated for virus infection. Medium devoid of virus was placed in toxicity control wells and cell control wells. Plates were incubated at 37° C. with 5% CO₂ until marked CPE (>80% CPE for most virus strains) is observed in virus control wells. The plates were then stained with 0.011% neutral red for approximately two hours at 37° C. in a 5% CO₂ incubator. The neutral red medium was removed by complete aspiration, and the cells rinsed 1× with phosphate buffered solution (PBS) to remove residual dye. The PBS was completely removed, and the incorporated neutral red eluted with 50% Sorensen's citrate buffer/50% ethanol for at least 30 minutes. Neutral red dye penetrates into living cells, thus, the more intense the red color, the larger the number of viable cells present in the wells. The dye content in each well was quantified using a spectrophotometer at 540 nm wavelength. The dye content in each set of wells was converted to a percentage of dye present in untreated control wells using a Microsoft Excel computer-based spreadsheet and normalized based on the virus control. The 50% effective (EC₅₀, virus-inhibitory) concentrations and 50% cytotoxic (CC₅₀, cell-inhibitory) concentrations were then calculated by regression analysis. The quotient of CC₅₀ divided by EC₅₀ gave the selectivity index (SI) value. Agents showing SI values≥10 are considered most active, though SI values of about 7 or 8 are also considered active. Exemplary results are shown in FIG. 2.

### EXAMPLE 2

### Reduction of SARS-Cov2 Virus Yield (Secondary VYR Assay)

A selection of agents considered active in primary testing were further tested in a confirmatory assay. This assay was set up similar to the methodology described above only eight half-log₁₀ concentrations of inhibitor were tested for antiviral activity and cytotoxicity. After sufficient virus replication occurs (3 days for SARS-CoV-2), a sample of supernatant was taken from each infected well (three replicate wells are pooled) and tested immediately or held frozen at −80° C. for later virus titer determination. After maximum CPE is observed, the viable plates were stained with neutral red dye. The incorporated dye content was quantified as described above to generate the EC₅₀ and CC₅₀ values.

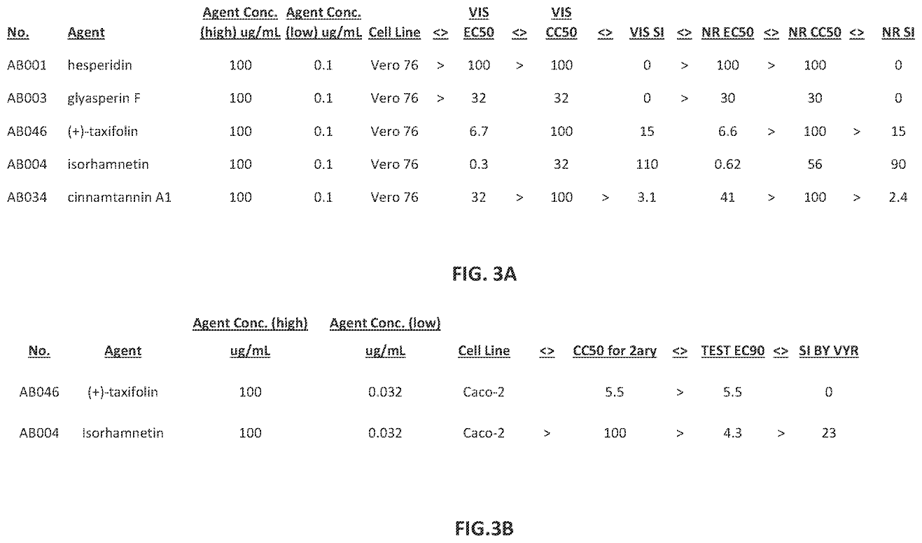

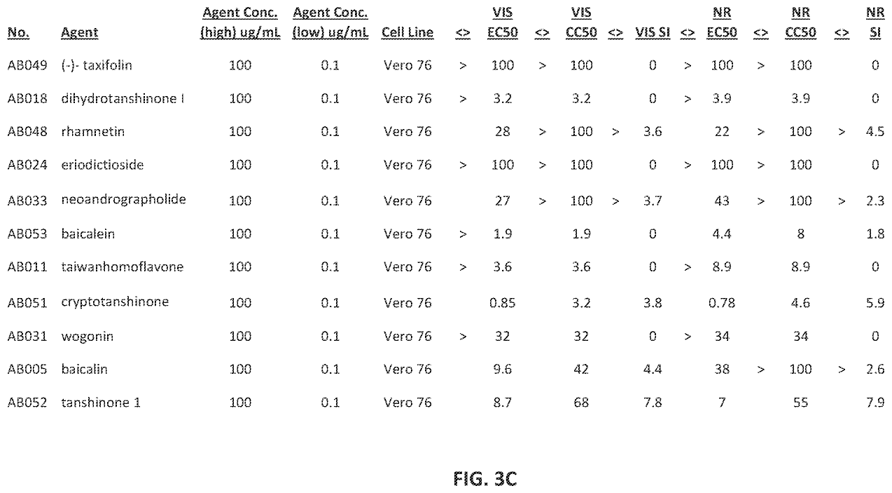

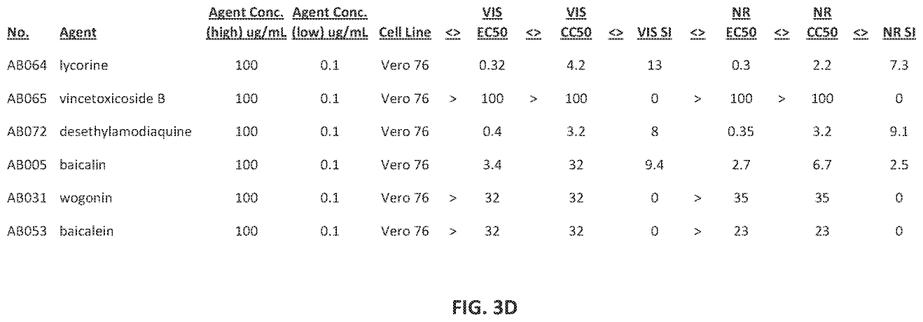

The VYR test is a direct determination of how much the test agent inhibits virus replication. Virus yielded in the presence of test compound was titrated and compared to virus titers from the untreated virus controls. Titration of the viral samples (collected as described in the paragraph above) was performed by endpoint dilution (Reed and Muench). Serial 1/10 dilutions of virus were made and plated into 4 replicate wells containing fresh cell monolayers of Vero 76 cells. Plates were then incubated, and cells scored for presence or absence of virus after distinct CPE is observed, and the CCID₅₀ calculated using the Reed-Muench method. The 90% (one log₁₀) effective concentration (EC₉₀) was calculated by regression analysis by plotting the log₁₀ of the inhibitor concentration versus log₁₀ of virus produced at each concentration. Dividing EC₉₀ by the CC₅₀ gives the SI value for this test. Exemplary results are shown in FIGS. 3A-3D.

### EXAMPLE 3

### Inhibition of SARS-CoV2 Infection Using a Single Agent

Artesunate, Dihydroartemisinin, and Isorhamnetin were tested to determine the effectiveness of SARS-CoV2 infection of human lung cells. Calu3 (ATCC, HTB-55) cells were pretreated with test agents for 2 hours prior to continuous infection with SARS-CoV-2 (isolate USA WA1/2020) at a MOI=0.5. Forty-eight hours post-infection, cells were fixed, immunostained, and imaged by automated microscopy for infection (dsRNA+ cells/total cell number) and cell number. Sample well data was normalized to aggregated DMSO control wells and plotted versus drug concentration to determine the IC50.

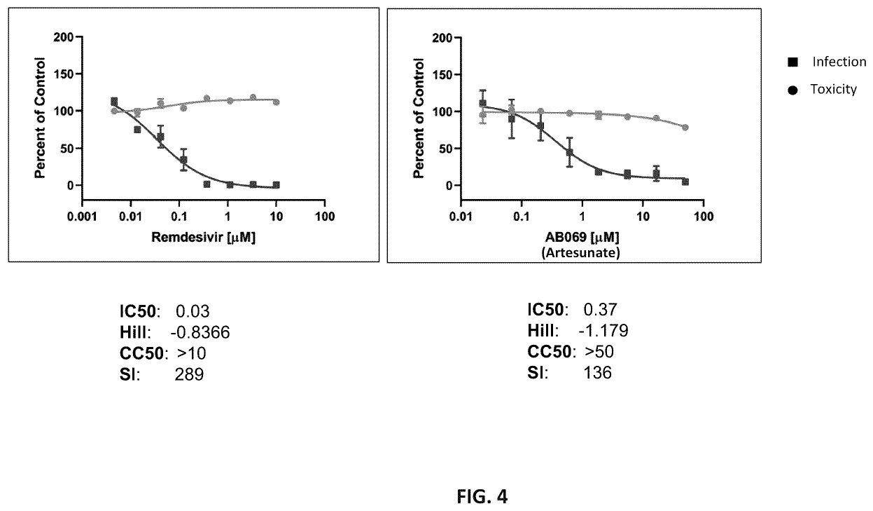

FIG. 4 depicts a resulting inhibition profile of SARS-CoV2 infection after pretreating Calu3 cells with Artesunate (AB069) compared to a Remdesivir. TABLE 2 summarizes the average viability and percent infection compared to DMSO control over three replicates (sample well measurement/aggregated DMSOavg)*100.

TABLE 2
SARS-CoV2 Inhibition with Artesunate

| Sample | Viability | % Infection |
|---|---|---|
| 0.02 μM | 95.4 | 110.9 |
| 0.07 μM | 102.6 | 90.0 |
| 0.21 μM | 100.5 | 80.7 |
| 0.62 μM | 97.7 | 44.7 |
| 1.85 μM | 95.1 | 18.0 |
| 5.56 μM | 92.9 | 14.7 |
| 16.67 μM | 91.1 | 16.1 |
| 50.00 μM | 78.3 | 4.7 |

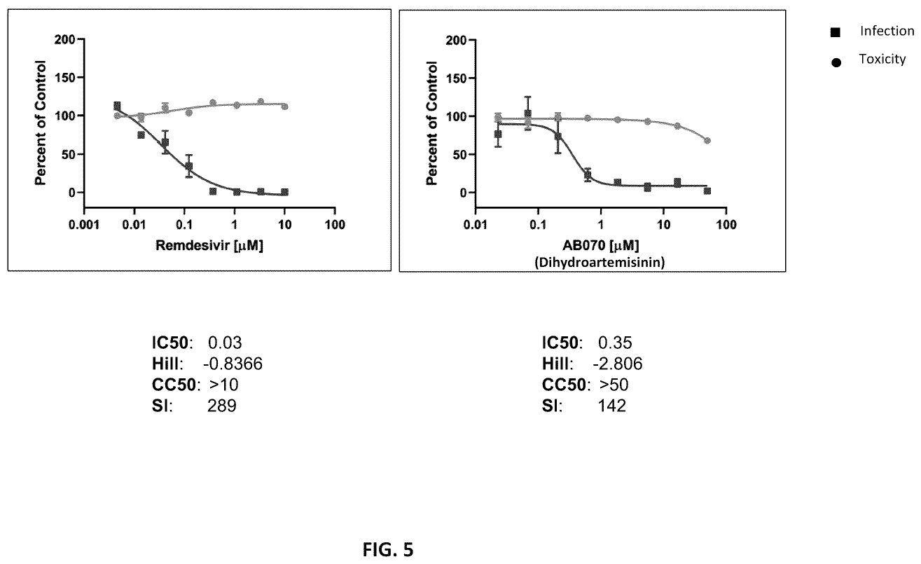

FIG. 5 depicts a resulting inhibition profile of SARS-CoV2 infection after pretreating Calu3 cells with Dihydroartemisinin (AB070) compared to a Remdesivir. TABLE 3 summarizes the average viability and percent infection compared to DMSO control over three replicates (sample well measurement/aggregated DMSOavg)*100.

TABLE 3
SARS-CoV2 Inhibition with Dihydroartemisinin

| Sample | Viability | % Infection |
|---|---|---|
| 0.02 μM | 98.6 | 76.6 |
| 0.07 μM | 91.6 | 103.6 |
| 0.21 μM | 99.3 | 73.6 |
| 0.62 μM | 97.7 | 23.0 |
| 1.85 μM | 95.4 | 13.5 |
| 5.56 μM | 93.3 | 7.0 |
| 16.67 μM | 87.4 | 12.8 |
| 50.00 μM | 68.2 | 2.0 |

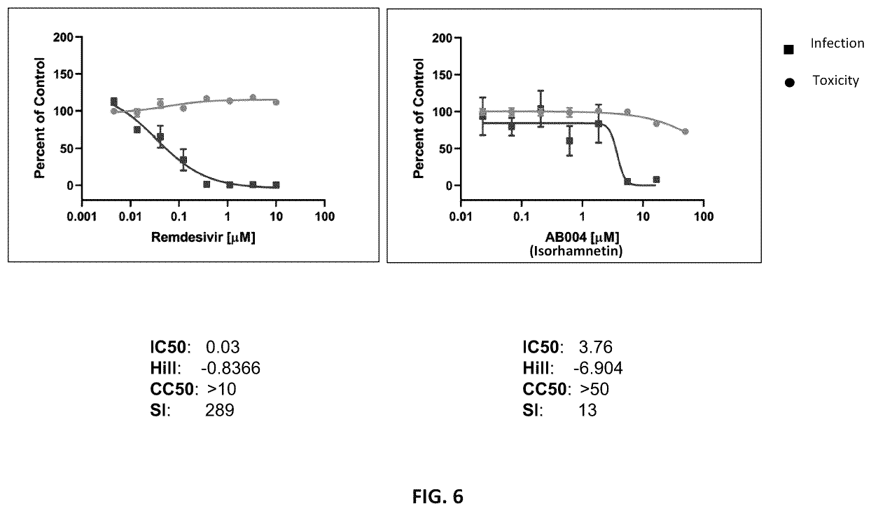

FIG. 6 depicts a resulting inhibition profile of SARS-CoV2 infection after pretreating Calu3 cells with Isorhamnetin (AB004) compared to a Remdesivir. TABLE 4 summarizes the average viability and percent infection compared to DMSO control over three replicates (sample well measurement/aggregated DMSOavg)*100.

TABLE 4
SARS-CoV2 Inhibition with Isorhamnetin

| Sample | Viability | % Infection |
|---|---|---|
| 0.02 μM | 99.6 | 93.6 |
| 0.07 μM | 99.6 | 79.5 |
| 0.21 μM | 99.4 | 103.8 |
| 0.62 μM | 98.8 | 60.4 |
| 1.85 μM | 101.0 | 83.8 |
| 5.56 μM | 99.8 | 5.3 |
| 16.67 μM | 83.7 | 7.9 |
| 50.00 μM | 73.0 | |

While the claimed subject matter has been described in terms of various embodiments, the skilled artisan will appreciate that various modifications, substitutions, omissions, and changes may be made without departing from the spirit thereof. Accordingly, it is intended that the scope of the subject matter limited solely by the scope of the following claims, including equivalents

1. A method for treating coronavirus disease 2019 (COVID-19) infection in a subject, the method comprising administering to the subject a therapeutically effective amount of an agent selected from one or more of the group consisting of artesunate, dihydroartemisinin, isorhamnetin, lycorine, (+)-taxifolin, tashinone I, and desethylamodiaquine.

2. (canceled)

3. The method of claim 1, wherein at least two of the agents are administered and target at least two different stages of a COVID-19 life cycle.

4. The method of claim 1, wherein at least two of the agents are administered.

5. The method of claim 1, further comprising administering to the subject a therapeutically effective amount of an additional agent that down regulates cytokine production, optionally TGF-beta.

6. The method of claim 1, further comprising administering to the subject a therapeutically effective amount of an additional agent that reduces or inhibits inflammation or pulmonary fibrosis in response to viral infection.

7. The method of claim 1, further comprising administering to the subject a therapeutically effective amount of an additional agent selected from the group consisting of camostat, remdesivir, molnupiravir, AT-527, artemisinin, nirmatrelvir and ritonavir, nirmatrelvir, ritonavir, baicalein, rhamnetin, (−)-taxifolin, and cryptotanshinone.

8. (canceled)

9. A pharmaceutical composition for the treatment of coronavirus disease 2019 (COVID-19), the composition comprising in a pharmaceutically acceptable carrier, two or more agents selected from the group consisting of artesunate, dihydroartemisinin, isorhamnetin, lycorine, (+)-taxifolin, baicalin, tashinone I, and desethylamodiaquine.

10. A pharmaceutical composition for the treatment of coronavirus disease 2019 (COVID-19), the composition comprising in a pharmaceutically acceptable carrier:
a first agent selected from the group consisting of artesunate, dihydroartemisinin, isorhamnetin, lycorine, (+)-taxifolin, tashinone I, and desethylamodiaquine; and
a second agent that is different from the first agent and is selected from the group consisting of artesunate, dihydroartemisinin, isorhamnetin, lycorine, (+)-taxifolin, tashinone I, desethylamodiaquine, camostat, remdesivir, molnupiravir, AT-527, artemisinin, nirmatrelvir and ritonavir, nirmatrelvir, ritonavir, baicalein, rhamnetin, (−)-taxifolin, and cryptotanshinone.
